(19) United States
(12) Patent Application Publication
(10) Pub. No.: US 2023/0355393 A1
(43) Pub. Date: Nov. 9, 2023

(54) INFLATABLE PENILE PROSTHESIS HAVING A CYLINDER WITH A CORE MEMBER

(71) Applicant: Boston Scientific Scimed, Inc., Maple Grove, MN (US)

(72) Inventors: James Ryan Mujwid, Hudson, WI (US); Abigail Rae Brooks, Saint Louis Park, MN (US); John Anders Bostrom, Minneapolis, MN (US)

(21) Appl. No.: 18/311,501

(22) Filed: May 3, 2023

Related U.S. Application Data
(60) Provisional application No. 63/364,227, filed on May 5, 2022.

(51) Int. Cl. A61F 2/26

(52) U.S. Cl. CPC A61F 2/26; A61F 2250/0003

(57) ABSTRACT

According to an aspect, an implant includes an inflatable member and a pump assembly. The pump assembly is configured to facilitate a transfer of a fluid to the inflatable member to place the inflatable member in an inflated configuration. The inflatable member has a sidewall that defines a lumen and a core member disposed within the lumen. The core member has a surface and defines a plurality of closed cells. The fluid is configured to be disposed adjacent the surface of the core member when the inflatable member is in the inflated configuration.

### CROSS-REFERENCE TO RELATED APPLICATION

This application claims priority to U.S. Provisional Patent Application No. 63/364,227, filed on May 5, 2022, entitled “INFLATABLE PENILE PROSTHESIS HAVING A CYLINDER WITH A CORE MEMBER”, the disclosure of which is incorporated by reference herein in its entirety.

### TECHNICAL FIELD

This disclosure relates generally to bodily implants and more specifically to bodily implants, such as penile prostheses that include inflatable members.

### BACKGROUND

One treatment for male erectile dysfunction is the implantation of a penile prosthesis that mechanically erects the penis. Some existing penile prostheses include inflatable cylinders or members that can be inflated or deflated using a pump mechanism. In some existing devices, the inflatable cylinder or member requires a relatively large amount of force to inflate. Additionally, in some existing devices, the pump mechanism may require many sequential squeezes or activations to inflate the cylinder or member. Furthermore, in some existing devices, the inflatable cylinder or member may assume a flat, unnatural shape when in a deflated configuration.

### SUMMARY

According to an aspect, an implant includes an inflatable member and a pump assembly. The pump assembly is configured to facilitate a transfer of a fluid to the inflatable member to place the inflatable member in an inflated configuration. The inflatable member has a sidewall that defines a lumen and a core member disposed within the lumen. The core member has a surface and defines a plurality of closed cells. The fluid is configured to be disposed adjacent the surface of the core member when the inflatable member is in the inflated configuration.

In some embodiments, the core member has a cylindrical shape. In some embodiments, the core member defines a lumen. In some embodiments, the core member has a cylindrical shape and defines a lumen. In some embodiments, the core member defines a lumen, the lumen being configured to receive the fluid when the inflatable member is in the inflated configuration.

In some embodiments, the fluid is configured to be disposed between the core member and the surface of the sidewall when the inflatable member is in the inflated configuration. In some embodiments, the fluid is configured to be disposed directly adjacent the sidewall and directly adjacent the surface of the sidewall when the inflatable member is in the inflated configuration.

In some embodiments, the sidewall is formed of a non-expandable material. In some embodiments, the sidewall is formed of a single layer of material. In some embodiments, the sidewall is formed of a single layer of non-expandable material.

In some embodiments, the device includes a reservoir operatively coupled to the pump assembly and configured to retain the fluid.

In some embodiments, the core member is formed of a closed cell silicon foam material. In some embodiments, the core member is formed of a closed cell urethane foam material.

In some embodiments, the inflatable member having a tubular shape when in the inflated configuration. In some embodiments, the inflatable member is configured to be placed in the inflated configuration and a deflated configuration, the inflatable member having a tubular shape when in the deflated configuration and having a tubular shape when in the inflated configuration.

According to another aspect, an implant includes an inflatable member, a reservoir and a pump assembly. The pump assembly is operatively coupled to the reservoir and to the inflatable member. The pump assembly is configured to facilitate a transfer of a fluid from the reservoir and to the inflatable member to place the inflatable member in an inflated configuration. The inflatable member has a sidewall that defines a lumen and a core member disposed within the lumen. The core member defines a plurality of closed cells. The fluid is configured to be disposed adjacent the core member and outside of the closed cells when the inflatable member is in the inflated configuration.

In some embodiments, the fluid is configured to be disposed directly adjacent the core member when the inflatable member is in the inflated configuration.

In some embodiments, the core member has a cylindrical shape. In some embodiments, the core member defines a lumen. In some embodiments, the core member defines a lumen, the lumen being configured to receive the fluid when the inflatable member is in the inflated configuration.

### BRIEF DESCRIPTION OF THE DRAWINGS

FIG. 1 schematically illustrates an implant according to an embodiment.

FIG. 2 illustrates an implant according to another embodiment.

FIG. 3 illustrates the implant of FIG. 2 implanted within a patient according to an embodiment.

FIG. 4 is a see-through view of the cylinder or inflatable member of the implant of FIG. 2.

FIG. 5 is a cross-sectional view of the cylinder or inflatable member of the implant of FIG. 2 while in a deflated configuration taken along line C-C of FIG. 3.

FIG. 6 is a cross-sectional view of the cylinder or inflatable member of the implant of FIG. 2 while in an inflated configuration taken along line C-C of FIG. 3.

FIG. 7 is a perspective view of a core member according to another embodiment.

FIG. 8 is a cross-sectional view of a cylinder or inflatable member with the core member of FIG. 7.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices such as penile prostheses or other bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human male, a human female, or any other mammal.

The embodiments discussed herein may improve the performance of an inflatable member of the device. For example, the inflatable member may have improved stiffness or rigidity, improved reliability, or improved deflation or inflation times. In some embodiments, inflating the inflatable member may be facilitated by requiring less force, less pressure, or less fluid transfer to inflate the inflatable member. Additionally, in some embodiments, the inflatable member may retain or maintain a tubular shape when the inflatable member is placed in its deflated configuration.

The embodiments may include an inflatable penile prosthesis having a pump assembly, an inflatable member, and a reservoir. The inflatable member may be implanted into the corpus cavernosae of a patient or user, the reservoir may be implanted in the user's abdomen, and the pump assembly may be implanted in the scrotum. The pump assembly may switch between an inflation position and a deflation position such that a user can operate the device to place the inflatable penile prosthesis in either an inflation mode to transfer fluid from the reservoir to the inflatable member or a deflation mode to transfer the fluid from the inflatable member back to the reservoir.

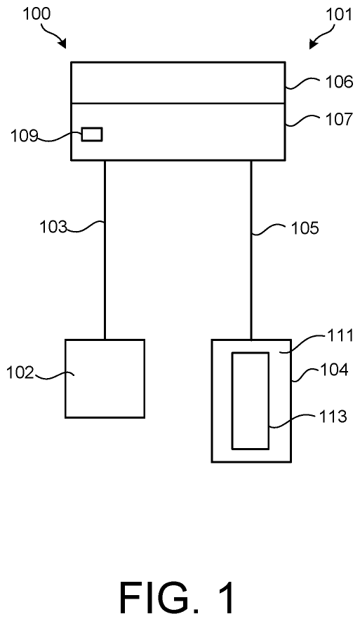

FIG. 1 schematically illustrates an inflatable implant 100 according to an aspect. In some embodiment, the implant 100 is a penile prosthesis. The inflatable implant 100 includes a reservoir 102, a cylinder or inflatable member 104, and a pump assembly 101 configured to transfer fluid between the reservoir 102 and the inflatable member 104. In some examples, the inflatable member 104 may be implanted into the corpus cavernosae of the user, the reservoir 102 may be implanted in the abdomen or pelvic cavity of the user (e.g., the reservoir 102 may be implanted in the lower portion of the user's abdominal cavity or the upper portion of the user's pelvic cavity), and the pump assembly 101 may be implanted in the scrotum of the user.

The inflatable member 104 may be capable of becoming more rigid and/or expanding upon the injection of fluid into a cavity of the inflatable member 104. For instance, upon injection of the fluid into the inflatable member 104, the inflatable member 104 may increase its length and/or width, as well as increase its rigidity. In some examples, the inflatable member 104 may include a pair of cylinders or at least two cylinders, e.g., a first cylinder member and a second cylinder member. The volumetric capacity of the inflatable member 104 may depend on the size of the cylinders. In some examples, the volume of fluid in each cylinder may vary from about 10 milliliters in smaller cylinders and to about 50 milliliters in larger sizes. In some examples, the first cylinder member may be larger than the second cylinder member. In other examples, the first cylinder member may have the same size as the second cylinder member.

In some embodiments, the inflatable member 104 or cylinder includes an outer sheath or tubular member 111 and a core member 113. In some embodiments, the outer sheath or tubular member 111 of the inflatable member 104 defines a lumen and the core member 113 is disposed within the lumen.

In some embodiments, the outer sheath or tubular member 111 includes a single layer of material. Additionally, in some embodiments, the outer sheath or tubular member 111 is formed of a non-expandable material. In other words, the outer sheath or tubular member 111 is configured to retain its dimensions and not stretch or expand when fluid is disposed within the cavity of the outer sheath or tubular member 111. In other embodiments, the outer sheath or tubular member 111 is formed of multiple layers or of multiple materials. For example, in some embodiments, the outer sheath or tubular member 111 may be formed of more than one layer. In some embodiments, the multi-layer outer sheath or tubular member 111 is configured to not expand.

In some embodiments, the core member 113 is formed of a closed-cell material. In other words, the core member 113 may be formed of a material that defines a plurality of cavities or cells that are closed or fluidically isolated from each other (they are not fluidically coupled to each other). In such embodiments, fluid may be disposed adjacent to the core member 113 and the fluid would not enter into the cavities or cells defined by the material that forms the core member. In some embodiments, the core member 113 is formed of a closed cell silicone foam material. In other embodiments, the core member 113 is formed of a closed cell urethane foam material. In yet other embodiments, the core member 113 is formed of a different material.

In some embodiments, the inflatable member 104 includes end caps or end tip members. The end tip members may be coupled to the end portions of the outer sheath. In some embodiments, the structural member is disposed within the lumen defined by the outer sheath and is disposed between the end caps or end tip members.

In some embodiments, the core member 113 may allow the inflatable member to be inflated at a relatively low pressure. In some embodiments, this may allow the user to inflate the inflatable member 104 with less pumps or activations of the pump or may allow the user to apply less force to the pump to inflate the inflatable member 104. Details of the pump assembly 101 are described below. Additionally, in some embodiments, the core member 113 of the inflatable member 104 may allow the inflatable member 104 to retain or maintain a tubular shape (or a more anatomically correct shape) when the inflatable member 104 is in its deflated configuration.

The reservoir 102 may include a container having an internal chamber configured to hold or house fluid that is used to inflate the inflatable member 104. The volumetric capacity of the reservoir 102 may vary depending on the size of the inflatable penile prosthesis 100. In some examples, the volumetric capacity of the reservoir 102 may be 3 to 150 cubic centimeters. In some examples, the reservoir 102 is constructed from the same material as the inflatable member 104. In other examples, the reservoir 102 is constructed from a different material than the inflatable member 104.

The inflatable implant 100 may include a first conduit connector 103 and a second conduit connector 105. Each of the first conduit connector 103 and the second conduit connector 105 may define a lumen configured to transfer the fluid to and from the pump assembly 101. The first conduit connector 103 may be coupled to the pump assembly 101 and the reservoir 102 such that fluid can be transferred between the pump assembly 101 and the reservoir 102 via the first conduit connector 103. For example, the first conduit connector 103 may define a first lumen configured to transfer fluid between the pump assembly 101 and the reservoir 102. The first conduit connector 103 may include a single or multiple tube members for transferring the fluid between the pump assembly 101 and the reservoir 102.

The second conduit connector 105 may be coupled to the pump assembly 101 and the inflatable member 104 such that fluid can be transferred between the pump assembly 101 and the inflatable member 104 via the second conduit connector 105. For example, the second conduit connector 105 may define a second lumen configured to transfer fluid between the pump assembly 101 and the inflatable member 104. The second conduit connector 105 may include a single or multiple tube members for transferring the fluid between the pump assembly 101 and the inflatable member 104. In some examples, the first conduit connector 103 and the second conduit connector 105 may include a silicone rubber material.

The pump assembly 101 may switch between an inflation mode in which the fluid in the reservoir 102 is transferred to the inflatable member 104 through the pump assembly 101 in a first direction (e.g., inflation direction) and a deflation mode in which the fluid in the inflatable member 104 is transferred back to the reservoir 102 through the pump assembly 101 in a second direction (e.g., deflation direction).

The pump assembly 101 includes a pump (also referred to as a pump bulb member) 106 and a valve body 107. The valve body 107 also includes a selection member 109. The selection member 109 may be used to select or change the mode in which the pump assembly is in. For example, the selection member 109 may be moved from a first position to a second position to place the device in its deflation mode. The selection member 109 may then be moved back to its first position to place the device in its inflation mode. In some embodiments, the selection member 109 is movable with respect to the valve body 107. For example, in some embodiments, the selection member 109 is slidably coupled or slidable with respect to the valve body 107.

The pump 106 may be squeezed or depressed by the user in order to facilitate the transfer of fluid from the reservoir 102 to the inflatable member 104. For example, in the inflation mode, while the user is operating the pump 106, the pump 106 may receive the fluid from the reservoir 102, and then output the fluid to the inflatable member 104. When the user switches to the deflation mode, at least some of the fluid can automatically be transferred back to the reservoir 102 (due to the difference in pressure from the inflatable member 104 to the reservoir 102). Then, the user may squeeze the inflatable member 104 to facilitate the further transfer of fluid through the pump 106 or pump assembly 101 to the reservoir 102.

In some examples, the pump 106 may include a flexible member defining a cavity. In some examples, the pump 106 may define a pump shell having a flexible bulb and a valve body connector, where the valve body connector is designed to fit at least partially over the valve body 107. In some examples, the pump 106 may include a squeeze pump. In some examples, the pump 106 may include a portion that is round or substantially round. In some examples, the pump 106 may include ribbing or dimples to aid the user in gripping the pump 106. The pump 106 may use suction and pressure to move the fluid in and out of the cavity of the pump 106 in the inflation mode. For example, the user may depress or squeeze the pump 106 to expel the fluid out of the cavity, and, when the flexible member returns to its original shape, the resulting suction pushes the fluid into the cavity of the pump 106. In some examples, the pump 106 may have a bulb spring rate that is designed to refill the pump 106 in a selected time frame.

As discussed above, the selection member 109 may be used to select or change the mode in which the pump assembly is in. For example, in one embodiment, the selection member 109 may be placed in the inflate position and the user may then operate the pump 106 to inflate the inflatable member 104 (i.e., move the fluid from the reservoir 102 to the inflatable member 104). For example, the user may repeatedly depress or squeeze the pump 106 until the desired rigidity is achieved.

In some examples, if the reservoir 102 is at least partially pressurized, the fluid may automatically flow out of the reservoir 102 and into the inflatable member 104 without the user depressing or squeezing the pump 106 until the pressure is at least partially equalized between the reservoir 102 and the inflatable member 104.

Then, when the user wants to deflate the inflatable member 104, the user moves selection member 109 to its deflated position. The user may then operate the pump 106 to deflate the inflatable member 104 (i.e., move the fluid from the inflatable member 104 to the reservoir 102). The pump 106 may then return to its original form, which provides a suction force causing fluid to be drawn into the pump 106 from the inflation member 104. The fluid from the inflation member 104 fills the pump 106 (or at least partially fills the pump 106). This pump cycle is repeated until the inflatable member 104 is deflated.

In some examples, the fluid may automatically (upon movement of the selection member 109 to its deflate position) flow out of the inflatable member 104 and into the reservoir 102 without the user depressing or squeezing the pump 106 until the pressure is at least partially equalized between the reservoir 102 and the inflatable member 104.

In some examples, after the inflation member 104 has been deflated, the pump 106 may be squeezed to place the pump in a contracted position or configuration.

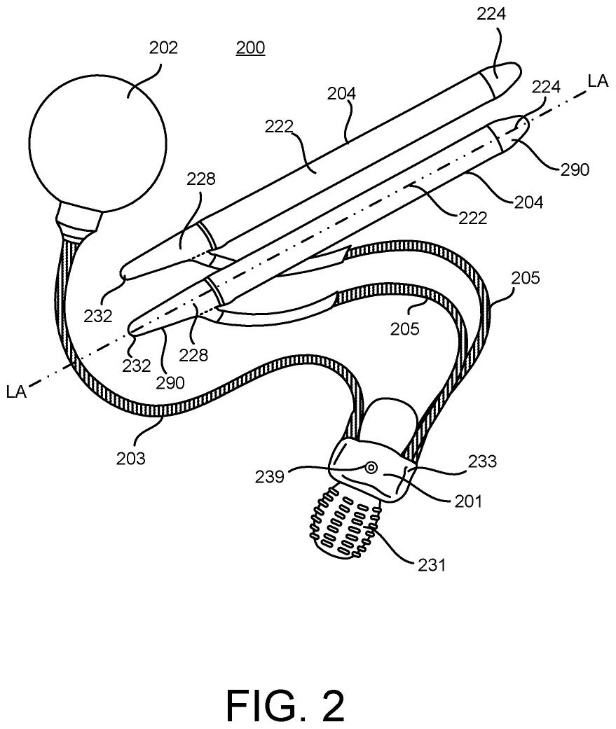

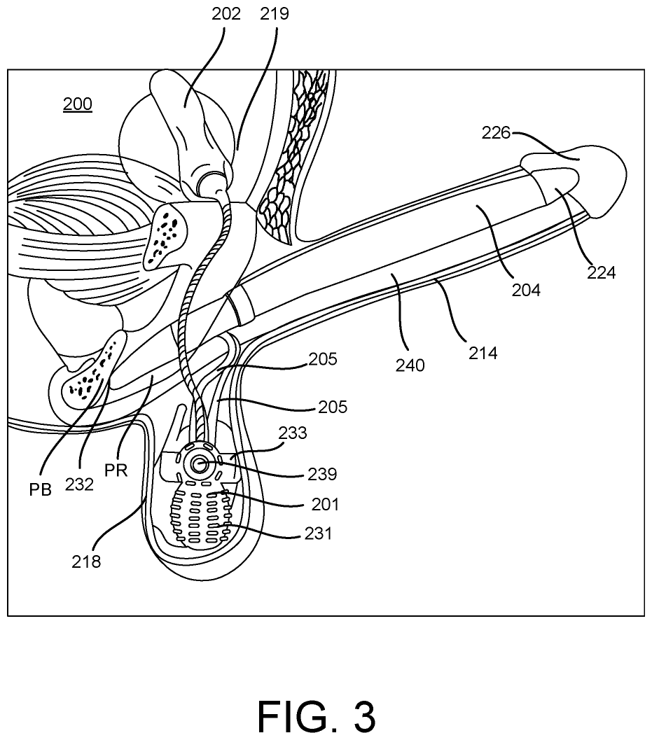

FIG. 2 illustrates an implant 200 according to an aspect. FIG. 3 schematically illustrates the implant 200 placed within a body of the user or patient.

In the illustrated embodiment, the implant 200 is a penile prosthesis. The implant 200 may include a pair of cylinders 204, and the pair of cylinders or inflatable members 204 are implanted in a penis 214. For example, one of the cylinders 204 may be disposed on one side of the penis 214. The other cylinder 204 (not shown in FIG. 3) of the pair of cylinders may be disposed on the other side of the penis 214. The cylinder 204 may include a second end portion 224, a cavity or inflation chamber 222, and a first end portion 228 having a rear tip 232.

The implant 200 includes a pump assembly 201, which may be implanted into the patient's scrotum 218. A pair of conduit connectors 205 may attach the pump assembly 201 to the pair of inflatable members or cylinders 204 such that the pump assembly 201 is in fluid communication with the pair of inflatable members or cylinders 204. Also, the pump assembly 201 may be in fluid communication with a reservoir 202 via a conduit connector 203. The reservoir 202 may be implanted into the user's abdomen 219. The inflation chamber or portion 222 of the cylinder 204 may be disposed within the penis 214. The second end portion 224 of the cylinder 204 may be at least partially disposed within the crown portion 226 or glands of the penis 214. The first end portion 228 may be implanted into the patient's pubic region PR with the rear tip 232 proximate the pubic bone PB.

In order to implant the inflatable members or cylinders 204, the surgeon first prepares the patient. The surgeon often makes an incision in the penoscrotal region, e.g., where the base of the penis 214 meets with the top of the scrotum 218. From the penoscrotal incision, the surgeon may dilate the patient's corpus cavernosae 240 to prepare the patient to receive the pair of inflatable members or cylinders 204. The corpus cavernosum is one of two parallel columns of erectile tissue forming the dorsal part of the body of the penis 214, e.g., two slender columns that extend substantially the length of the penis 214. The surgeon will also dilate two regions of the pubic area (first corpora cavernosae) to prepare the patient to receive the first end portion 228. The surgeon may measure the length of the first and second corpora cavernosae from the incision and the dilated region of the pubic area to determine an appropriate size of the inflatable members or cylinders 204 to implant.

After the patient is prepared, the implant 200 is implanted into the patient. The second tip of the second end portion 224 of each cylinder 204 may be attached to a suture. The other end of the suture may be attached to a needle member (e.g., Keith needle). The needle member is inserted into the incision and into the dilated corpus cavernosum. The needle member is then forced through the crown or glands of the penis 226. The surgeon tugs on the suture to pull the cylinder 204 into the corpus cavernosum. This is done for each cylinder of the pair of cylinders 204. Once the inflation chamber 222 is in place, the surgeon may remove the suture from the second tip. The surgeon then inserts the first end portion 228. The surgeon inserts the rear end of the cylinder 204 into the incision and forces the first end portion 228 toward the pubic bone PB until each cylinder 204 is in place.

In the illustrated embodiment, each of the inflatable members or cylinders 204 is structurally and functionally similar. Accordingly, only one of the inflatable members or cylinders will be discussed in detail.

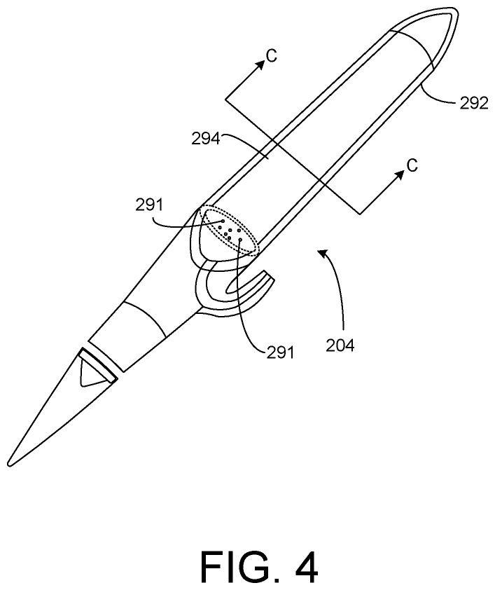

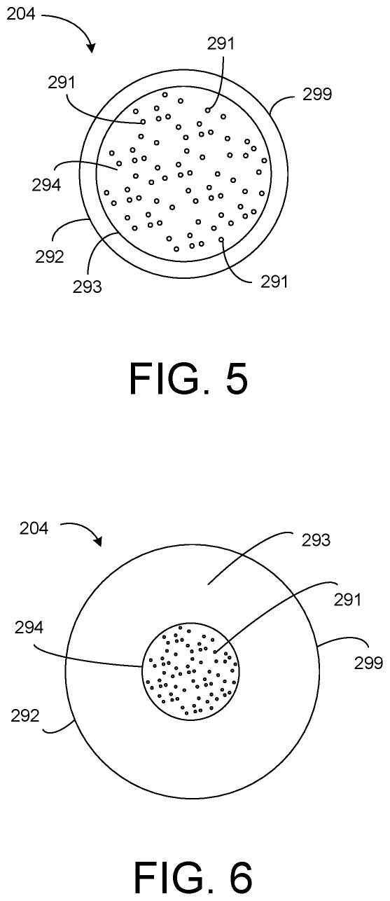

FIG. 4 is a see-through view of the cylinder or inflatable member of the implant 200. FIG. 5 is a cross-sectional view of the cylinder or inflatable member 204 of the implant 200 while in a deflated configuration taken along line C-C of FIG. 3. FIG. 6 is a cross-sectional view of the cylinder or inflatable member 204 of the implant 200 while in an inflated configuration taken along line C-C of FIG. 3.

The inflatable member 204 may be configured to be more rigid when in the inflated configuration than when in the deflated configuration. Additionally, the inflatable member 204 may be capable of expanding upon the injection of fluid into a cavity of the inflatable member 204. For instance, upon injection of the fluid into the inflatable member 204, the inflatable member 204 may increase its length and/or width, as well as increase its rigidity. The volumetric capacity of the inflatable member 204 may depend on the size of the cylinders. In some examples, the volume of fluid in each cylinder may vary from about 10 milliliters in smaller cylinders and to about 50 milliliters in larger sizes.

In the illustrated embodiment, the inflatable member or cylinder 204 includes an outer sheath or tubular member 292 and a core member 294. The outer sheath or tubular member 292 of the inflatable member 204 has a sidewall 299 and defines a lumen 293. The core member 294 is disposed within the lumen 293.

In the illustrated embodiment, the outer sheath or tubular member 292 includes a single layer of material. Additionally, in the illustrated embodiment, the outer sheath or tubular member 292 is formed of a non-expandable material. In other words, the outer sheath or tubular member 292 is configured to retain its dimensions and/or not stretch or expand when fluid is disposed within the lumen 293 of the outer sheath. In some embodiments, the outer sheath 292 is formed of a fabric material, such as a non-expanding fabric material.

In the illustrated embodiment, the core member 294 is formed of a closed-cell material. In other words, the core member 294 may be formed of a material that defines a plurality of cavities or cells 291 that are closed or fluidically isolated from each other (they are not fluidically coupled to each other). In the illustrated embodiment, fluid may be disposed adjacent to the core member 294 and the fluid will not enter into the cavities or cells defined by the material that forms the core member. In some embodiments, the core member 294 is formed of a closed cell silicone foam material. In other embodiments, the core member 294 is formed of a closed cell urethane foam material. In yet other embodiments, the core member 294 is formed of a different material.

As best illustrated in FIGS. 4-6, the core member 294 has a tubular shape and is elongated. The core member 294 is disposed within the lumen 293 and extends within the lumen 293 defined by the outer sheath 292 along the longitudinal axis of the outer sheath 292. In some embodiments, the core member 294 is configured to compress or be compressed when the inflatable member is placed in the inflated configuration. Specifically, as best illustrated in FIG. 5, the core member 294 has a size, such as a diameter D1, when the inflatable member 204 is in the deflated configuration. As best illustrated in FIG. 6, the core member 294 has a size, such as a diameter D2, when the inflatable member 204 is in the inflated configuration. As the core member 294 compresses when the inflatable member 204 is in the inflated configuration, the diameter D1 is larger than the diameter D2.

As best illustrated in FIG. 6, when the inflatable member 204 is placed in the inflated configuration, the fluid is disposed between a surface of the core member 294 and the outer sheath 292. The fluid does not enter the cells or cavities 291 defined by the core member 294.

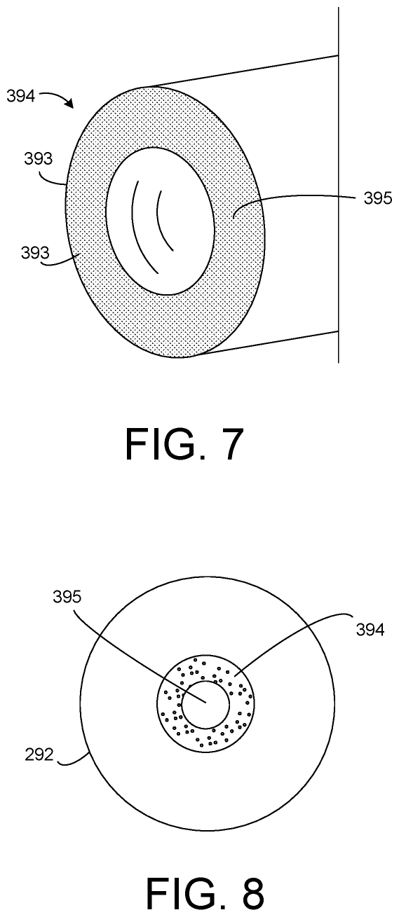

While the core member 294 has a tubular or rod shape, in other embodiments, the core member may be of different shapes. FIG. 7 is a perspective view of a core member 394 according to another embodiment. The core member 394 has a cylindrical shape and defines a lumen 395. The lumen 395 extends from one end of the core member 394 to another end of the core member 394.

The core member 394 is formed of a closed-cell material. In other words, the core member 394 may be formed of a material that defines a plurality of cavities or cells that are closed or fluidically isolated from each other (they are not fluidically coupled to each other). In the illustrated embodiment, fluid may be disposed adjacent to the core member 394 and the fluid will not enter into the cavities or cells defined by the material that forms the core member. In some embodiments, the core member 394 is formed of a closed cell silicone foam material. In other embodiments, the core member 394 is formed of a closed cell urethane foam material. In yet other embodiments, the core member 394 is formed of a different material.

The core member 394 may be disposed within the lumen defined by the outer sheath. As best illustrated in FIG. 8, when the inflatable member is placed in the inflated configuration, the fluid is disposed between a surface of the core member 394 and the outer sheath 292. The fluid is also configured to be disposed within the lumen 395 defined by the core member 394. The fluid does not enter the cells or cavities 393 defined by the core member 394.

In some embodiments, the core member 294 (or core member 394) may allow the inflatable member 204 to be inflated at a relatively low pressure. In some embodiments, this may allow the user to inflate the inflatable member 204 with less pumps or activations of the pump or may allow the user to apply less force to the pump to inflate the inflatable member 204. Details of the pump assembly 201 are described below. Additionally, in some embodiments, the core member 294 of the inflatable member 204 may allow the inflatable member 204 to retain or maintain a tubular shape (or a more anatomically correct shape) when the inflatable member 204 is in its deflated configuration.

The pump assembly 201 may switch between an inflation mode in which the fluid in the reservoir 202 is transferred to the inflatable member 204 (or inflatable members) through the pump assembly 201 in a first direction (e.g., inflation direction) and a deflation mode in which the fluid in the inflatable member 204 (or inflatable members) is transferred back to the reservoir 202 through the pump assembly 201 in a second direction (e.g., deflation direction).

The pump assembly 201 includes a pump bulb member or pump 231, a valve body 233, and a selection member 239. The selection member may be used to select or change the mode in which the pump assembly 201 is in. For example, the selection member 239 may be moved from a first position to a second position to place the device in its deflation mode. The selection member 239 may then be moved back to its first position to place the device in its inflation mode. In some embodiments, the selection member 239 is movable with respect to the valve body 233. For example, the selection member 239 may be slidably coupled or slidable with respect to the valve body 233. In some embodiments, the selection member 239 includes stop members, such as shoulders or detents that engage members of the valve body 233 to lock or help retain the selection member 239 in one of its first and second positions. In other embodiments, the selection member 239 may be disposed or coupled to another portion of the device.

The pump 231 may be squeezed or depressed by the user in order to facilitate the transfer of fluid from the reservoir 202 to the inflatable member 204. For example, in the inflation mode, while the user is operating the pump 231, the pump 231 may receive the fluid from the reservoir 202, and then output the fluid to the inflatable member 204. When the user switches to the deflation mode, at least some of the fluid can automatically be transferred back to the reservoir 202 (due to the difference in pressure from the inflatable member 204 to the reservoir 202). Then, the user may squeeze the inflatable member 204 to facilitate the further transfer of fluid through the pump 231 to the reservoir 202.

Then, when the user wants to deflate the inflatable members 204, the user moves selection member 239 to its deflated position. The user may then operate the pump 231 to deflate the inflatable members 204 (i.e., move the fluid from the inflatable members 204 to the reservoir 202). For example, the user may repeatedly depress or squeeze the pump 231 until the deflation is completed. The pump 231 may then return to its original form, which provides a suction force causing fluid to be drawn into the pump 231 from the inflation members 204. The fluid from the inflation members 204 fills the pump 231 (or at least partially fills the pump 231). This pump cycle is repeated until the inflatable members 204 are deflated.

In some examples, the fluid may automatically (upon movement of the selection member 239 to its deflate position) flow out of the inflatable member 204 and into the reservoir 202 without the user depressing or squeezing the pump 231 until the pressure is at least partially equalized between the reservoir 202 and the inflatable member 204.

In some examples, after the inflation member 204 has been deflated, the pump 231 may be squeezed to place the pump in a contracted position or configuration.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implant, comprising:
an inflatable member; and
a pump assembly configured to facilitate a transfer of a fluid to the inflatable member to place the inflatable member in an inflated configuration,
the inflatable member having a sidewall that defines a lumen and a core member disposed within the lumen, the core member having a surface and defining a plurality of closed cells, the fluid being configured to be disposed adjacent the surface of the core member when the inflatable member is in the inflated configuration.

2. The implant of claim 1, wherein the core member has a cylindrical shape.

3. The implant of claim 1, wherein the core member defines a lumen.

4. The implant of claim 1, wherein the core member has a cylindrical shape and defines a lumen.

5. The implant of claim 1, wherein the core member defines a lumen, the lumen being configured to receive the fluid when the inflatable member is in the inflated configuration.

6. The implant of claim 1, wherein the fluid is configured to be disposed between the core member and the surface of the sidewall when the inflatable member is in the inflated configuration.

7. The implant of claim 1, wherein the fluid is configured to be disposed directly adjacent the sidewall and directly adjacent the surface of the sidewall when the inflatable member is in the inflated configuration.

8. The implant of claim 1, wherein the sidewall is formed of a non-expandable material.

9. The implant of claim 1, wherein the sidewall is formed of a single layer of material.

10. The implant of claim 1, wherein the sidewall is formed of a single layer of non-expandable material.

11. The implant of claim 1, further comprising:
a reservoir operatively coupled to the pump assembly and configured to retain the fluid.

12. The implant of claim 1, wherein the core member is formed of a closed cell silicone foam material.

13. The implant of claim 1, wherein the core member is formed of a closed cell urethane foam material.

14. The implant of claim 1, wherein the inflatable member having a tubular shape when in the inflated configuration.

15. The implant of claim 1, wherein the inflatable member is configured to be placed in the inflated configuration and a deflated configuration, the inflatable member having a tubular shape when in the deflated configuration and having a tubular shape when in the inflated configuration.

16. An implant, comprising:
an inflatable member;
a reservoir; and
a pump assembly operatively coupled to the reservoir and to the inflatable member, the pump assembly being configured to facilitate a transfer of a fluid from the reservoir and to the inflatable member to place the inflatable member in an inflated configuration,
the inflatable member having a sidewall that defines a lumen and a core member disposed within the lumen, the core member defining a plurality of closed cells, the fluid being configured to be disposed adjacent the core member and outside of the closed cells when the inflatable member is in the inflated configuration.

17. The implant of claim 16, wherein the fluid is configured to be disposed directly adjacent the core member when the inflatable member is in the inflated configuration.

18. The implant of claim 16, wherein the core member has a cylindrical shape.

19. The implant of claim 16, wherein the core member defines a lumen.

20. The implant of claim 16, wherein the core member defines a lumen, the lumen being configured to receive the fluid when the inflatable member is in the inflated configuration.
